(19) United States
(12) Patent Application Publication
(10) Pub. No.: US 2003/0129624 A1
(43) Pub. Date: Jul. 10, 2003

(54) Methods of preparing amplified nucleic acid molecules

(75) Inventors: Eric Eastman, Gaithersburg, MD (US); Glenn Hoke, Gaithersburg, MD (US); John Hartwell, Gaithersburg, MD (US); Larry Millstein, McLean, VA (US); Michael Kuziora, Gaithersburg, MD (US); Richard Guilfoyle, Gaithersburg, MD (US)

Correspondence Address: MORGAN LEWIS & BOCKIUS LLP, 1111 PENNSYLVANIA AVENUE NW, WASHINGTON, DC, 20004, US

(73) Assignee: GENE LOGIC, INC.

(21) Appl. No.: 10/244,595

(22) Filed: Sep. 17, 2002

Related U.S. Application Data
(63) Continuation of application No. PCT/US01/08501, filed on Mar. 16, 2001.
(63) Continuation-in-part of application No. 09/669,739, filed on Sep. 26, 2000, now abandoned.
(60) Provisional application No. 60/190,056, filed on Mar. 17, 2000.

(51) Int. Cl. C12Q 1/68; C12P 19/34

(52) U.S. Cl. 435/6; 435/91.2

(57) ABSTRACT

New and improved methods are provided for generating amplified nucleic acid molecules from cellular mRNA. The methods are robust and reliable, and can be used to provide gene fragments for use in methods of analyzing gene expression patterns.

### CROSS REFERENCE TO RELATED APPLICATIONS

This application claims the benefit of U.S. Provisional Application Ser. No. 60/190,056, filed Mar. 17, 2000, and U.S. patent application Ser. No. 09/669,739, filed Sep. 26, 2002, both of which are specifically incorporated herein by reference in their entireties.

### BACKGROUND OF THE INVENTION

1. Field of the Invention

The present invention provides new and improved methods for generating amplified nucleic acid molecules. The methods, which can be used to amplify both DNA and RNA molecules, are robust and reliable and can be used to provide RNA gene fragments for use in methods of analyzing gene expression patterns.

2. Description of the Related Art

In recent years, methods have been developed for the analysis of gene expression in individual cells and tissues. These methods are providing powerful insights into the cellular processes that occur, for example, in disease states. For example, the gene expression profile for normal and diseased cells can be compared to provide information regarding the identity of genes whose expression levels are modified in the disease state. This information can provide insights that are useful in developing treatments for the disease, or in understanding the pathology of the disease.

Microfabricated arrays of large numbers of oligonucleotide probes, called “DNA chips” offer great promise for a wide variety of applications. In particular DNA chips are useful for generating gene expression profiles of the type discussed above. Typically, DNA chip technology involves a microarray containing many thousands of unique DNA probes attached to a solid support. Mixtures containing fragments of target nucleic acids derived from the cells or tissues of interest are applied to the chip, and fragments that hybridize with the probes are retained on the chip while fragments that do not hybridize are washed away. The success of DNA chip technology, however, depends on the ability to obtain sufficient amounts of labeled single stranded target nucleic acid molecules of an appropriate size that can be hybridized to the chips. Moreover, the amounts of the simgle-stranded mucleic acid molecules should reflect the amount of the corresponding mRNA in the cell or tissue of interest if the gene expression analysis is to provide any useful quantitative information.

It is often desirable to fragment the target nucleic acid molecule prior to hybridization with a probe array, in order to provide segments which are more readily accessible to the probes, which hybridize more rapidly, and which avoid secondary structures and/or hybridization to multiple probes. On the other hand, target molecules that are too short are more likely not to hybridize or to hybridize in a non-specific manner, providing an inaccurate assessment of gene expression patterns. RNA molecules can be fragmented in a straightforward manner by heating in a solution of basic pH or under other suitable conditions and, accordingly, RNA is often the nucleic acid of choice for generating gene fragments for use in methods of gene expression analysis.

Obtaining sufficient mRNA for the study of gene expression often is problematic. Typically, amplification of the mRNA in some fashion is required to provide sufficient material for detection. Linear amplification methods are preferred over exponential amplification methods such as PCR because they provide a more accurate representation of the relative abundance of expressed genes in a given cell or tissue, preserving rare sequences and providing more accurate quantitation.

U.S. Pat. No. 5,545,522, (Van Gelder et al.,) describes a method in which mRNA molecules are reverse-transcribed using a complementary primer linked to an RNA polymerase promoter region to make a first strand cDNA. Second strand synthesis relies upon self-priming either by the formation of a hairpin loop at the 3′ end of the first strand of cDNA or from short stretches of RNA molecules that remain after RNase H treatment. Following second strand synthesis, anti-sense RNA (aRNA) is transcribed from the cDNA by introducing an RNA polymerase capable of binding to the promoter region. The resulting aRNA can be fragmented as described above.

This method has the disadvantage of relying either on the formation of the hairpin loop at the end of the first cDNA strand to prime second strand synthesis or on the unreliable nature of RNase H treatment to generate short stretches RNA fragments for priming. For example, first strand cDNA does not always reliably generate such a hairpin loop, meaning that second strand synthesis does not occur, generation of a double stranded promoter region does not occur, and therefore no aRNA molecule can be generated. Alternatively, RNase H treatment may not always provide reproducible stretches of RNA primers.

It is apparent, therefore, that a need exists for improved methods of generating amplified RNA molecules and RNA fragments that are representative of the type and amounts of cellular mRNA. Preferably, the overall methodologies will be capable of amplifying a broad range of target molecule without prior cloning and without knowledge of mRNA sequence. The present invention fulfills these and other needs.

### SUMMARY OF THE INVENTION

It is therefore an object of the present invention to provide improved methods for generating amplified RNA molecules and RNA fragments that can be used in gene expression analysis and other applications.

In accomplishing these objects, there has been provided, in accordance with one aspect of the present invention, a process for amplifying a population of RNA molecules, comprising the steps of preparing first strand cDNA molecules by reverse transcription using a primer molecule or plurality of primer molecules that hybridizes to the population or RNA molecules and wherein the primer molecule or plurality of primer molecules contains an upstream promoter sequence or region that is recognized by an RNA polymerase; synthesizing a double-stranded cDNA from the first strand cDNA, wherein synthesis of the second cDNA strand of the double-stranded cDNA is primed by an oligonucleotide mixture having random sequence; and transcribing copies of RNA initiated from the double stranded promoter region.

In one embodiment, the transcribed copies of RNA are subjected to a second round of amplification by converting RNA copies generated by a first amplification to cDNA and performing a second round of in vitro transcription to convert the cDNA into RNA.

There also has been provided, in accordance with another aspect of the invention, a method for amplifying a population of RNA molecules, comprising the steps of preparing a first strand cDNA molecule by reverse transcription using a primer molecule that hybridizes to the RNA molecule wherein the primer molecule contains an upstream nucleotide sequence that is recognized by a restriction endonuclease having a 6, 7, or 8 base recognition sequence, synthesizing a double stranded cDNA from the first strand cDNA, wherein synthesis of the second cDNA strand of the double stranded cDNA is primed by an oligonucleotide mixture having random sequence; digesting the double stranded cDNA with a restriction endonuclease that recognizes the upstream nucleotide sequence to provide a double stranded cDNA containing a cohesive terminus; ligating a double stranded promoter oligonucleotide to the cohesive terminus, wherein the promoter oligonucleotide comprises a promoter region that is recognized by a RNA polymerase; and transcribing copies of RNA initiated from the promoter region.

In one embodiment, the promoter region can operably be recognized by a T bacteriophage RNA polymerase, such as a T3, T7 or SP6 bacteriophage RNA polymerase.

In another embodiment, the RNA is eukaryotic mRNA, preferably mRNA having a poly(A) tail.

In still another embodiment, the aRNA molecules are fragmented. The fragmentation can be performed via heat and/or treatment at high pH, for a time sufficient to cleave at least about 95% of the RNA molecules.

In yet another embodiment, the nucleotides incorporated in the transcription step are labeled with a detectable label. The detectable label may be at least one of a radioisotope, a chromophore, a fluorophore, an enzyme, a reactive group or an affinity ligand.

In embodiments of the invention the oligonucleotide mixture having a random sequence comprises oligonucleotide mixtures selected from the group consisting of a tetramer oligonucleotide mixture, a pentamer oligonucleotide mixture, a hexamer oligonucleotide mixture, a heptamer oligonucleotide mixture, an octamer oligonucleotide mixture, a nonamer oligonucleotide mixture and a decamer oligonucleotide mixture (i.e., 4, 5, 6, 7, 8, 9, and 10 nucleotides). The oligonucleotide mixture may be selected from the group consisting of a hexamer oligonucleotide mixture, a heptamer oligonucleotide mixture, an octamer oligonucleotide mixture and a nonamer oligonucleotide mixture (i.e., 6, 7, 8 and 9 nucleotides). In more preferred embodiments, the oligonucleotide mixture may be selected from the group consisting of a hexamer oligonucleotide mixture and a nonamer oligonucleotide mixture (i.e., 6 and 9 nucleotides). Most preferably the oligonucleotide mixture is a nonamer oligonucleotide mixture.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE PREFERRED EMBODIMENT

In accordance with the present invention, novel methods are provided for the generation of amplified nucleic acid molecules. In particular, there are provided methods for generating amplified anti-sense RNA molecules that correspond in sequence and in relative amount to cellular mRNA molecules. That is, the methods provide amplified anti-sense RNA (hereinafter “aRNA”) comprising a sequence that is substantially complementary to a sequence found in a cellular mRNA molecule or in a population of cellular mRNA molecules. Moreover, when applied to populations or mixtures of cellular mRNA molecules, the amplification methods of the invention provide aRNA molecules in relative quantities that reflect the relative quantities of those cellular mRNA molecules. In particular, the methods provide gene fragments in a quantity and form suitable for gene expression analysis.

The target nucleic acid population for the practice of this invention may be isolated from a cellular source using many available methods well-known in the art. For example, for RNA isolation, the Chomczynski method, e.g., isolation of total cellular RNA using guanidine isothiocyanate (described in U.S. Pat. No. 4,843,155) may be used or commercial kits such as are available from Qiagen and Rneasy may be used as well. Alternatively, the starting material may be mRNA isolated using, for example, oiigo-dT streplavidin beads by methods that are well known in the art.

In general, the methods involve an amplification process that generates aRNA by transcription from a double-stranded cDNA that comprises a recognition sequence for an RNA polymerase. In a first method first strand cDNA synthesis is carried out by reverse transcription using a primer that recognizes the cellular mRNA molecule. The primer contains a promoter region that can be recognized by an RNA polymerase. The skilled artisan is well aware of methods of carrying out reverse transcription reactions. See, for example, Sambrook et al., (1989), Molecular Cloning: A Laboratory Manual Second Edition, (Cold Spring Harbor). In one embodiment, the recognition by the primer occurs via recognition of the poly(A) tail at the 3′ end of the mRNA molecules, i.e. a poly(dT)-containing primer is used. Use of a poly(dT)-containing primer in this fashion means that first strand synthesis will occur from essentially all cellular poly(A)-containing mRNA molecules. This is useful if the amplified RNA is intended to be used for studying the complete gene expression profile for the cell or tissue from which the RNA was derived. When a more limited gene expression profile is of interest, for example, when the expression profile of a gene family is of interest, the first strand primer can be designed to recognize a nucleotide sequence that is conserved within the gene family. For example, it is known that G-protein-coupled receptors contain regions of conserved sequence that can be used to design primers or primer mixtures that allow selective isolation of cDNAs encoding the receptors. Alternatively, primers specific for single genes also can be used, alone or in combination. Methods of designing gene-specific primers and primers that recognize conserved gene family sequences are well known in the art.

Upstream (to the 5′ end) of the primer sequence that recognizes the mRNA molecule, the primer also contains a promoter sequence for an RNA polymerase. Preferably, the promoter sequence is one that is recognized by a bacteriophage RNA polymerase such as a T bacteriophage (for example T3 or T7), or SP6 RNA polymerase. A preferred primer containing a promoter sequence is the T7 promoter-containing primer:

5′-ggc cag tga att gta ata cga ctc act ata ggg agg cgg ttt ttt ttt ttt ttt ttt ttt ttt-3′ (SEQ ID NO: 1).

After completion of first strand synthesis, second strand synthesis then is primed using a mixture of random primers of defined length. The random primer mixture contains a stochastic mixture of all possible nucleotide sequences for a given length of primer. The primer mixtures are conveniently prepared using methods of solid-phase oligonucleotide synthesis or by hydrolysis of larger sequences that are well known in the art. Instruments for preparing oligonucleotides are commercially available from, for example, PE Biosystems (Foster City, Calif.). Also, oligonucleotides can be purchased from commercial vendors such as Life Technologies (Rockville, Md.) or Midland Certified Reagent Company (Midland, Tex.). Preferably the random primers contained in the mixture all have the same length (contain the same number of nucleotides), although the skilled artisan will recognize that mixtures of nucleotides having different lengths may be used.

In particular, the limitations imposed by currently available methods of oligonucleotide synthesis mean that the lengths of the primers contained in any synthesized stochastic mixture likely will be somewhat heterogeneous. For example, a mixture of putative nonamers likely will contain a small amount of primers containing 8 or fewer nucleotides. It is also possible that, depending upon the source of the primer mixture, a mixture of putative nonamers will contain a small amount of primers containing 10 or more nucleotides. If desired, the amount of these shorter and/or longer molecules can be reduced or eliminated by purification of the primer mixture, for example, by HPLC or by gel purification. Such purification methods are well known in the art. The purification methods will normally achieve a nucleotide length distribution within the oligonucleotide mixture wherein at least 90% of the oligonucleotides in the mixture are the specified length. For example, an oligonucleotide mixture containing nine nucleotides (nonamers) as defined herein has at least 90% of the oligonucleotides in the mixture having the specified length of nine nucleotides and is referred to herein as a “nonamer oligonucleotide mixture”. An oligonucleotide mixture containing six nucleotides (hexamers) as defined herein has at least 90% of the oligonucleotides in the mixture having the specified length of six nucleotides and is referred to herein as a “hexamer oligonucleotide mixture”. Of course higher nucleotide length distributions on the order of at least 95%, at least 98%, or 99% of the oligonucleotides in the mixture having the specified length may be achieved and are within the scope of the invention. The cost and difficulty in obtaining such purities are to be weighed against the benefit obtained by such purities.

For efficient priming of second strand synthesis, oligonucleotide mixtures or primers containing at least six nucleotides (hexamers) preferably are used, although the skilled artisan will recognize that shorter primers, such as tetramers and pentamers, also can be used. Longer primers, for example, heptamers, octamers, nonamers and decamers also can be used. However, the statistical likelihood of any particular primer being complementary to a given sequence within an mRNA molecule drops off exponentially with the addition of each extra nucleotide. Thus, it has been calculated for a primer containing 14 nucleotides that there is a statistical likelihood that it will be complementary to only one sequence within the entire human genome. Moreover, the complexity of a stochastic or random primer mixture also increases exponentially as the length of the nucleotides increase.

Mixtures of oligonucleotide mixtures can also be used, for example hexamers can be combined with any one or more of tetramers, pentamers, heptamers, octamers, nonamers and decamers. Similarly, nonamers may be combined with any one or more of tetramers, pentamers, hexamers, heptamers, octamers, and decamers.

The skilled artisan will recognize, therefore, that primers containing more than 6 nucleotides can be used in the present invention, but that such mixtures become increasingly complex, and any particular primer becomes statistically less likely to recognize a sequence within a given mRNA molecule. Surprisingly, the present inventors have discovered that random mixtures of oligonucleotides containing nine nucleotides (nonamers) provide unexpectedly superior results to those obtained using mixtures of hexamers.

Synthesis of second strand cDNA is achieved by addition of a template-dependent DNA polymerase, such as E. coli DNA polymerase. This produces double-stranded cDNA containing a double-stranded promoter sequence corresponding to the promoter sequence present in the first strand primer.

First strand cDNA also can be generated from a DNA molecule. For example, double-stranded DNA can be heat denatured and a gene-specific promoter-containing primer can be used to prime first strand synthesis using a DNA polymerase. Second strand synthesis is then carried out as described above.

In an alternative embodiment, first strand cDNA synthesis from RNA is carried out by reverse transcription using a primer that contains a sernes of nucleotides comprising one strand of a recognition sequence for a “rare cutter” restriction endonuclease. This sequence is present upstream of the primer sequence that is complementary to the mRNA molecule. As described above, the primer can recognize a poly(A) tract, can recognize a gene family, of can be gene specific. A “rare cutter” restriction endonuclease is an endonuclease with a recognition sequence that is at least six, and preferably at least seven or eight nucleotides long. The endonuclease NotI is an example of a rare cutter endonuclease.

Second strand synthesis is then carried out using random priming as described above to produce double-stranded cDNA, where second strand synthesis provides a double-stranded recognition sequence for the rare cutter restriction endonuclease. The double-stranded cDNA then is digested with the rare cutter endonuclease and a DNA fragment containing a promoter sequence is ligated to the cohesive termini generated by the digestion. The promoter sequence preferably is a bacteriophage promoter of the type described above.

The double-stranded cDNA produced by these methods can be used directly for in vitro transcription, or it can first be purified. In vitro transcription is carried out by addition of an RNA polymerase that recognizes the promoter regions present in the double-stranded cDNA. Methods of carrying out in vitro transcription are well known in the art. In particular, when a commercially available RNA polymerase is used, the transcription carried out according to the manufacturer's instructions This transcription step also provides a convenient way to label the resulting transcribed RNA (aRNA) by incorporation of labeled nucleotides (e.g., radiolabeled or biotin-labeled) in the transcription reaction as described in more detail below. The resulting aRNA molecules can be fragmented as desired using heat and/or pH using methods that are well known in the art. The transcription reaction can be carried out until the desired number of aRNA copies are produced. Typically, for gene expression analysis, at least about 50 aRNA copies are produced for each molecule of double-stranded cDNA.

The present inventors have found that the best results are obtained by optimizing the amount of starting RNA and the ratio of RNA to added second strand primer. For mRNA preparations, 1-5 μg of poly(A)+ RNA is used (5 μg is optimum) for first strand synthesis, and for second strand 0.0015 to 3.0 μg per μg mRNA of primer, such as a hexamer mixture, is added (0.3 μg per μg is optimal within this range). For total RNA (which contains structural RNA plus mRNA) more starting RNA is used, e.g. 5-40 μg, typically 25-30 μg, and 0.1 to 3.0 μg per μg starting RNA second strand primer is used (0.3 μg per μg is optimal within this range).

In embodiments of the invention, enzyme concentrations may vary according to the particular enzyme, the ratio of oligonucletide to starting RNA, process temperatures as well as other factors. Enzyme concentrations are within the range of from 0.5 to 10.0 μl[10U/,μl] enzyme for a DNA ligase and from 2.0 to 40.0 μl[10 U/μl] enzyme for a DNA polymerase.

A divalent cation co-factor such as MgCl₂ may be used in second strand synthesis in concentrations of from 0.1 to 2.0 co-factor/enzyme where the MgCl₂ co-factor concentration is [50 mM] and the enzyme concentration (DNA ligase or DNA polymerase) is [10U/μl].

Incubation temperatures for second strand synthesis may range from 10° C. to 25° C., and preferably between 15° C. to 20° C.

Isolation of mRNAs and Synthesis of Double-Stranded cDNAs

The mRNAs are converted to cDNA by reverse transcriptase, e.g., oligo(dT)-primed first strand cDNA synthesis by reverse transcriptase, followed by second strand synthesis using a DNA polymerase such as DNA Polymerase I. Such methods are well-known to the skilled artisan. For general description of these methods, please see Sambrook et al., 1989, Molecular Cloning—A Laboratory Manual, 2nd ed., Vol. 1-3; and Ausubel et al., 1989, Current Protocols in Molecular Biology, Green Publishing Associates and Wiley Interscience, N.Y. When desired, the skilled artisan will recognize that primers specific for gene families can be used to provide cDNA mixtures containing a desired gene family.

In preparing the first strand cDNA, the primer is contacted with the mRNA in the presence of a reverse transcriptase and other reagents necessary for primer extension under conditions sufficient for first strand cDNA synthesis, where additional reagents include: dNTPs; buffering agents, e.g. Tris-Cl; cationic sources, both monovalent and divalent, e.g. KCl, MgCl₂; RNAase inhibitor and sulfhydryl reagents, e.g. dithiothreitol; and the like. A variety of enzymes, usually DNA polymerases, possessing reverse transcriptase activity can be used for the first strand cDNA synthesis step. Examples of suitable DNA polymerases include the DNA polymerases derived from organisms selected from the group consisting of a thermophilic bacteria and archaebacteria, retroviruses, yeasts, insects, primates and rodents. Preferably, the DNA polymerase will be selected from the group consisting of Moloney murine leukemia virus (M-MLV) or modified M-MLV reverse transcriptase lacking RNaseH activity, human, T-cell leukemia virus type I(HTLV-1), bovine leukemia virus (BV), Rous sarcoma virus (RSV), human immunodeficiency virus (HIV) and Thermus aquaticus (Taq) or Thermus thermophilus (Tth), avian reverse transcriptase, and the like. Suitable DNA polymerases possessing reverse transcriptase activity may be isolated from an organism, obtained commercially or obtained from cells which express high levels of cloned genes encoding the polymerases by methods known to those of skill in the art, where the particular manner of obtaining the polymerase will be chosen based primarily on factors such as convenience, cost, availability and the like.

The order in which the reagents are combined may be modified as desired. One protocol that may be used involves the combination of all reagents except for the reverse transcriptase on ice, then adding the reverse transcriptase and mixing at around 4° C. Following mixing, the temperature of the reaction mixture is raised to 37°-42° C. or higher temperatures, followed by incubation for a period of time sufficient for first strand cDNA primer extension product to form, usually about 1 hour.

Following first strand cDNA synthesis, the mixture of second strand primers (including but not limited to either hexamer or nonamer mixtures) are added and the subsequent reaction mixture heated to 95° C. for one minute followed by rapid cooling to 4° C.

First strand synthesis produces a mRNA/cDNA hybrid, which is then converted to double-stranded (ds) cDNA.

Typically the second strand cDNA reaction is carried out using 30 μl 5× second strand buffer (Life Technologies), 3.0 to 4.5 μl [10mM] dNTP mix, with 4.5 μl being optimum, 1.0 to 5 μl [10U/μl] E. Coli DNA ligase (life Technologies) with 5.0 μl being optimum, 4.0 to 20 μl [10U/μl] E. Coli DNA polymerase I, with 20 μl being optimum, 6.0 to 7.5 μl [50 mM] MgCl₂, with 7.5 μl being optimum, and DEPC treated water added to bring the final volume to 150 μl. The reaction is carried out for two hours at 16°-19° C., with 19° C. being optimum. To the mixture, 2.0 μl [10U/μl] T4 DNA polymerase is added and the resultant mixture is incubated for 5′ at 16° C. The reaction is stopped by the addition of 10 μl 0.5 M EDTA pH 8.0.

Incorporation of Labels into the Amplification Product

According to a preferred embodiment of the invention, the aRNA molecules are labeled, by any of many methods well-known in the art, with a marker for easy detection. The labeled fragments are particularly desired for many purposes in biotechnology, such as for the analysis of gene expression patterns and determination of DNA polymorphism.

As used herein, the terms “label” or “labeled” refers to incorporation of a detectable marker, e.g., by incorporation of a radioactively or non-radioactively labeled nucleotide. Various methods of labeling RNA molecules are known in the art and may be used.

Labeling of the aRNA according to the present invention may be achieved by incorporating a marker-labeled nucleotide into the transcription product. A large portion of available labeling method currently in use are radioactive and they can be obtained from a wide variety of commercial sources. Examples of radiolabels include, but are not restricted to, ³²P, ³H, ¹⁴C, or ³⁵S.

A large number of convenient and sensitive non-isotopic markers are also available. In general, all of the non-isotopic methods of detecting hybridization probes that are currently available depend on some type of derivitization of the nucleotides to allow for detection, whether through antibody binding, or enzymatic processing, or through the fluorescence or chemiluminescence of an attached “reporter” molecule. The aRNA product labeled with non-radioactive reporters incorporate single or multiple molecules of the label nucleotide which contain the reporter molecule, generally at specific cyclic or exocycilc positions.

Techniques for attaching reporter groups have largely relied upon (a) functionalization of 5′ or 3′ termini of the monomeric nucleosides by numerous chemical reactions (see Cardullo et al. (1988) Proc. Nat'l. Acad. Sci. 85: 8790-8794); (b) synthesizing modified nucleosides containing (i) protected reactive groups, such as NH₂, SH, CHO, or COOH, (ii) activatable monofunctional linkers, such as NHS esters, aldehydes, or hydrazides, or (iii) affinity binding groups, such as biotin, attached to either the heterocyclic base or the furanose moiety.

According to one aspect of the invention, the labeled nucleotide(s) are labeled with fluorogens. Examples of fluorogens include fluorescein and derivatives, isothiocyanate, dansyl chloride, phycoerythrin, allo-phycocyanin, phycocyanin, rhodamine, Texas Red, SYBR-Green or other proprietary fluorogens. The fluorogens are generally attached by chemical modification. The fluorogens can be detected by a fluorescence detector.

In a preferred embodiment, the labeled nucleotide can alternatively be labeled with a ligand to provide an enzyme or affinity label. For example, a nucleotide may have biotinyl moieties that can be detected by labeled avidin or streptavidin (e.g., streptavidin containing a fluorescent marker or enzymatic activity that can be detected by optical or calorimetric methods). The enzyme can be peroxidase, alkaline phosphatase or another enzyme giving a chromogenic or fluorogenic reaction upon addition of an appropriate substrate. For example, additives such as 5-amino-2,3-dihydro-1,4-phthalazinedione (also known as LUMINOL) (Sigma Chemical Company, St. Louis, Mo.) and rate enhancers such as p-hydroxybiphenyl (also known as p-phenylphenol) (Sigma Chemical Company, St. Louis, Mo.) can be used to amplify enzymes such as horseradish peroxidase through a luminescent reaction; and luminogeniec of fluorogenic dioxetane derivatives of enzyme substrates can also be used.

Usually, the labeled target nucleic acids comprises a direct label, such as a fluorescent label, radioactive label, or enzyme-conjugated label that catalyzes the conversion of a chromogenic substrate to a chromophore. However, it is possible, and often desirable for signal amplification, for the labeled binding component to be detected by at least one additional binding component that incorporates a label. Signal amplification can be accomplished by layering of reactants where the reactants are polyvalent.

Double in vitro Transcription Reaction

According to a preferred embodiment of the invention, amplified aRNAs are subjected to a second round of amplification. In one embodiment, aRNAs are converted to cDNA by reverse transcriptase, followed by second strand synthesis using a DNA polymerase such as DNA Polymerase I.

In one embodiment, first strand cDNA synthesis by reverse transcriptase is random-primed. In a preferred embodiment, synthesis is primed by an oligonucleotide mixture having random sequence that comprises oligonucleotides having a length selected from the group consisting of 4, 5, 6, 7, 8, 9, and 10 nucleotides. Alternatively, the oligonucleotide mixture having random sequence may consist essentially of hexamers or nonamers.

The use of reverse transcriptase during a second round of first strand cDNA synthesis may, lead to small cDNA products, which in turn may lead to small RNA products transcribed from these cDNA products during a second round of in vitro transcription. To increase the size of cDNA products, in another embodiment, E. coli DNA polymerase may be added during the second round of reverse transcription. The addition of E. coli DNA polymerase, with its 5′-3′ exonuclease activity, may lead to the generation of longer products.

In another embodiment, an oligo-dT primer is used to prime second strand synthesis. Preferably, the primer is the same primer used during the first round of cDNA synthesis. In a preferred embodiment the primer contains a promoter. Preferably, the promoter sequence is one that is recognized by a bacteriophage RNA polymerase such as a T bacteriophage (for example T3 or T7), or SP6 RNA polymerase. A preferred primer containing a promoter sequence is the T7 promoter-containing primer: 5′-ggc cag tga att gta ata cga ctc act ata ggg agg cgg ttt ttt ttt ttt ttt ttt ttt ttt-3′ (SEQ ID NO: 1).

In vitro transcription is carried out by addition of an RNA polymerase that recognizes the promoter sequences present in the double-stranded cDNA.

The use of a double in vitro transcription reaction enables the generation of a greater amount of aRNA from less input RNA. This facilitates the use of a smaller samples comprising fewer cells, including but not limited to cells derived from small tissue samples, micro-dissection techniques, or tissue or cell culture for use in methods of analyzing gene expression patterns. In one embodiment, the use of a sample comprising fewer cells facilitates the analysis of a more specific or homogeneous population of cells.

In a preferred embodiment, the sample comprises about 1,000 cells. In another embodiment, the sample comprises about 10,000 cells. In other embodiments, the sample comprises at least 10, at least 100 cells, or at least 1,000 cells. In a further embodiment, the sample comprises at least 1 cell as disclosed in U.S. Pat. No. 5,514,545 the disclosure of which is incorporated herein by reference. In other embodiments, the sample comprises 1-10, 10-100, 100-1,000, 1,000-10,000, or 10,000-100,000 cells, and all numbers subsumed within these ranges.

In a further embodiment, cells are obtained trom small tissue samples including but not limited to needle biopsies, or laser capture micro-dissected tissues.

### EXAMPLE 1

### cDNA Synthesis from Total RNA Using Random Hexamer Primers

The procedure described below uses the following reagents (suppliers shown in parentheses): 5′-phosphorylated, random hexamers (Operon); RNeasy Mini Kit (Qiagen); β-mercaptoethanol (Sigma); Ethanol (200 proof) (Wamer-Graham); 5× First Strand Buffer (Life Technologies, Gaithersburg, Md.); 5× Second Strand Buffer (Life Technologies, Gaithersburg, Md.); E-coli DNA Polymerase I (Life Technologies, Gaithersburg, Md.); 10 mM dNTPs (Life Technologies, Gaithersburg, Md.); E-coli DNA Ligase (Life Technologies, Gaithersburg, Md.) (optional component); Super Script II (Life Technologies, Gaithersburg, Md.); T4 DNA Polymerase (Life Technologies, Gaithersburg, Md.); T7-T(24) Primer (Operon); EDTA 0.5M (Life Technologies, Gaithersburg, Md.); 0.2 ml Thermowell tubes (Costar); PLG Tubes (1.5 ml) (5′ to 3′ Inc.); Phenol: Chloroform: Isoamyl Alcohol (25:24:1) (Amersco); Ammonium Acetate 5M (Sigma); Glycogen (Ambion); DEPC H₂O (Quality Biological).

A. RNA Isolation

RNA was isolated using the RNeasy kit (Qiagen) using the manufacturer's recommended conditions. This method is suitable for isolating up to 100 μg of RNA, which is the binding limit of the RNeasy mini spin column. The buffer RLT was warmed to dissolve any precipitate, then β-mercaptoethanol (B-ME) (10 μl per 1 ml of Buffer RLT) was added before use. 4 volumes of 100% EtOH also was added to Buffer RPE before initial use. The sample (lysed and digested cells or tissue that is deproteinated and delipidated) was adjusted to 100 μg nucleic acid/100 μl using RNase-free H₂O. If the sample was more than 130 μl, it was split into two tubes, and each was diluted to 100 μl with RNAsecure. Samples were placed into a 1.5 ml tube(s), and 350 μl of Buffer RLT was added, with mixing. Then 250 μl of 100% EtOH was added with mixing by pipetting. The sample (approx. 700 μl) was added to the RNeasy Column, which was centrifuged (spun) at room temperature for 15 seconds at 10,000 rpm.

The sample from the collection tube was reapplied to the same column, respun for 15 seconds at 10,000 rpm, and transferred to a new collection tube. 500 μl of buffer RPE was added and the sample spun at room temperature for 15 seconds at 10,000 rpm to wash. An additional 500 μl of buffer RPE was added to the column, which was spun at maximum speed to dry the membrane within the column

The column was transferred to a new 1.5 ml collection tube, and 30 μl of DEPC H₂O was added directly onto the membrane. After a 5 minute incubation the sample was spun for 1 minute at 10,000 rpm to elute. The eluate (30 μl) was added back to the column and spun again at 10,000 rpm. The OD of the final eluate was determined and the ratio of absorbance at 260 and 280 nm (“280/260 ratio”) was determined and used to calculate the concentration of RNA using standard methods. This sample was diluted to 1 mg/ml using DEPC water. If the concentration of the RNA was too low, it first was precipitated using standard methods followed by redilution to 1 mg/ml.

First Strand cDNA Synthesis

The table shown below was used to determine how much reverse transcriptase (Superscript II) was used for a given amount of total RNA:

| Total RNA (μg) | Superscript II RT (μl) 200 U/μl |
|---|---|
| 5.0 to 8.0 | 1.0 |
| 8.1 to 16.0 | 2.0 |
| 16.1 to 24.0 | 3.0 |
| 24.1 to 32.0 | 4.0 |
| 32.1 to 40.0 | 5.0 |

DEPC water, 100 picomolar T₇ promoter containing primer (ggc cag tga att gta ata cga ctc act ata ggg agg cgg ttt ttt ttt ttt ttt ttt ttt ttt) (SEQ ID NO: 1), and total RNA were sequentially added to a 0.2 ml thermocycler tube (to a final volume of 12 μl) with mixing and the samples incubated at 70° C. for 10 minutes, followed by chilling on ice. Then 7 μl of the following MASTER MIX was added to the mixture:

Mix: 4 μl of 5×1^{st} Strand Buffer

2 μl of 0.1M DTT

1 μl [10 mM] dNTP mix

The resulting mixture was mixed and incubated at 42° C., followed by addition of Superscript II RT (SSRT II). The sample was mixed well and incubated for 1 hour at 42° C.

Second Strand Synthesis Procedure (Example 1)

The sample was placed on ice, and 1.5 μg of random hexamers per μg of initial total RNA was added. The sample was mixed, spun, and incubated at 95° C. for 5 min, followed by rapid cooling to 4° C. After chilling for one minute, the sample was spun at 4° C. to collect condensation. A master mix was prepared containing the following:

30 μl 5× Second strand Buffer

3 μl [10 mM] dNTP Mix

1 μl [10U/μl] E. coli DNA Ligase

4 μl [10U/μl] E. coli DNA Polymerase I

DEPC H₂O to a volume of 130 μl minus the second strand primer volume

This master mix was added to the first strand synthesis reaction, which was annealed previously to second strand primers, and the sample incubated a 16° C. for 2 hours. Then 2 μl [10U] of T4 DNA Polymerase was added, and the sample cooled for 5 minutes at 16° C., followed by addition of 10 μl 0.5M EDTA.

Second Strand Synthesis Procedure (Optimized Example 1A)

The sample was placed on ice, and 0.3 μg of random hexamers per μg of initial total RNA was added. The sample was mixed, spun, and incubated at 95° C. for 1 min, followed by rapid cooling to 4° C. After chilling for one minute, the sample was spun at 4° C. to collect condensation. A master mix was prepared containing the following:

30 μl 5× Second strand Buffer

4.5 μl [10 mM] dNTP Mix

5 μl [10U/μl] E. coli DNA Ligase (optional)

20 μl [10U/μl] E. coli DNA Polymerase I

7.5 μl [50 mM] MgCl₂

DEPC H₂0 to a volume of 130 μl minus the second strand primer volume

This master mix was added to the first strand synthesis reaction, which was annealed previously to second strand primers, and the sample incubated a 19° C. for 2 hours. Then 2 μl [10U] of T4 DNA Polymerase was added, and the sample cooled for 5 minutes at 16° C., followed by addition of 10 μl 0.5M EDTA.

These samples were purified as follows:

cDNA Clean-Up Procedure (Using Phase Lock Gel (PLG) Tubes from 5′-3′ Inc.)

The samples were added to a PLG tube and an equal volume of (25:24:1) Phenol: Chloroform:Isoamyl Alcohol added (approximately 162 μl) for a final volume of 324 μl. The sample was mixed by inverting. The sample was spun at maximum speed for 2 minutes. The aqueous upper phase was transferred to a fresh 1.5 ml tube and ½ volume of 7.5M ammonium acetate, 2 μl of glycogen, and 2½ volumes of cold 100% EtOH were added to the sample, followed by vortexing. The sample was immediately centrifuged at >12,000 g at 4° C. for 20 minutes. The supernatant was removed and the precipitate washed with 500 μl of cold 80% EtOH, followed by centrifuging at RT for 5 minutes at maximum speed. The supernatant was again removed and the precipitate washed with 500 μl of cold 80% EtOH, followed by centrifuging at RT for 5 minutes at maximum speed.

The supernatant was removed and the pellet air dried for approx. 15 minutes. The pellet was resuspended in a small volume of DEPC H₂O using the table below to calculate the correct volume of water:

| Starting Total RNA (μg) | Volume to resuspend cDNA in (μl) |
|---|---|
| 5.0 to 8.0 | 2.4 |
| 8.1 to 16.0 | 4.8 |
| 16.1 to 24.0 | 7.3 |
| 24.1 to 32.0 | 9.6 |
| 32.1 to 40.0 | 12.0 |

This sample was checked by running a small aliquot (0.5 to 1 μl) on a 1.2% agarose gel, and used for in vitro transcription.

The optimized Example 1A improved (increased) the ratio of longer second strands/shorter second strands when compared to the un-optimized Example 1.

### EXAMPLE 2

### cDNA Synthesis from mRNA Using Random Hexamer Primers

Total cellular RNA was prepared as described above, and mRNA was isolated using oligo(dT)-coated beads by standard methods. Sources for reagents was as described in Example 1. The amount of poly(A)+mRNA used was 1-5 μg, with amounts close to 5 μg being preferred.

The total volume of the first strand cDNA synthesis was 12 μl, and the ratio of SuperScript II to mRNA was always 200U per μg of mRNA.

First Strand Synthesis Procedure DEPC water, T₇-(T)₂₄ primer, and total RNA were sequentially added to a 0.2 ml thermocycler tube (to a final volume of 12 μl) with mixing and the samples incubated at 70° C. for 10 minutes, followed by chilling on ice. Then 7 μl of the following MASTER MIX was added to the mixture:

Mix: 4 μl of 5×1^{st} Strand Buffer

2 μl of 0.1M DTT

1 μl [10 mM] dNTP mix

The resulting mixture was mixed and incubated at 37° C., followed by addition of Superscript II RT (SSRT II). The sample was mixed well and incubated for 1 hour at 37° C.

Second Strand Synthesis Procedure (Example 2)

Random hexamers (3 μl of 50 ng/μl) were added per μg of first strand cDNA (assuming 100% synthesis efficiency). The reaction mixture was heated for 1 min at 95° C. and quickly chilled on a water-ice slurry. A master mix of the following was prepared:

30 μl 5× Second strand Buffer

3 μl [10 mM] dNTP Mix

1 μl [10U/μl] E. coli DNA Ligase (optional)

4 μl [10U/μl] E. coli DNA Polymerase I

To this mix was added DEPC H₂O so that total volume of 2^{nd} strand master mix plus 1^{st} strand/hexamer reaction mixture equalled 150 μl total volume. The 2^{nd} Strand master mix was added to the First Strand/Hexamer reaction mix and incubated at 16° C. for 2 hours. T4 DNA polymerase (2 μl [10U]) was added and the reaction cooled for 5 minutes at 16° C. EDTA (10 μl 0.5M) was added. The sample was then purified as described in Example 1 using PLG tubes. Briefly, the entire cDNA sample to the PLG tube, an equal volume of (25:24:1) Phenol:Chloroform:Isoamyl Alcohol (Approximately 162 μl) was added for a final volume of 324 μl. The tube was mixed by inversion and then spun at maximum speed for 2 minutes. The aqueous upper phase was transferred to a fresh 1.5 ml tube and ½ volume of 7.5M ammonium acetate, 2 μl of glycogen, and 2½ volume of cold 100% EtOH was added to the sample, which then was vortexed. The tube was immediately centrifuged at >12,000×g at 4° C. for 20 minutes. The supernatant was removed and washed with 500 μl of cold 80% EtOH. The tube was centrifuged at RT for 5 minutes at maximum speed and the supernatant removed. The tube was washed with 500 μl of cold 80% EtOH and centrifuge at RT for 5 minutes at maximum speed. The supernatant was removed and the pellet air dried for approx. 15 minutes. The pellet was resuspended in 1.8 μl of DEPC H₂O per μg mRNA and used for in vitro transcription as described below.

Second Strand Synthesis Procedure (Example 2A—optimized)

Random hexamers (0.3 μg of 1 μg starting mRNA) were added to the first strand reaction. The reaction mixture was heated for 1 min at 95° C. and quickly chilled on a water-ice slurry. A master mix of the following was prepared:

30 μl 5× Second strand Buffer

4.5 μl [10 mM] dNTP Mix

5 μl [10U/μl] E. coli DNA Ligase (optional)

20 μl [10U/μl] E. coli DNA Polymerase I

7.5 μl [50 mM] MgCl₂

To this mix was added DEPC H₂O so that total volume of 2^{nd} strand master mix plus 1^{st} strand/hexamer reaction mixture equalled 150 μl total volume. The 2^{nd} Strand master mix was added to the First Strand/Hexamer reaction mix and incubated at 19° C. for 2 hours. T4 DNA polymerase (2 μl [10U]) was added and the reaction cooled for 5 minutes at 16° C. EDTA (10 μl, 0.5M) was added. The sample was then purified as described in Example 1 using PLG tubes. Briefly, the entire cDNA sample to the PLG tube, an equal volume of (25:24:1) Phenol:Chloroform:Isoamyl Alcohol (Approximately 162 μl) was added for a final volume of 324 μl. The tube was mixed by inversion and then spun at maximum speed for 2 minutes. The aqueous upper phase was transferred to a fresh 1.5 ml tube and ½ volume of 7.5M ammonium acetate, 2 μl of glycogen, and 2½ volume of cold 100% EtOH was added to the sample, which then was vortexed. The tube was immediately centrifuged at >12,000×g at 4° C. for 20 minutes. The supernatant was removed and washed with 500 μl of cold 80% EtOH. The tube was centrifuged at RT for 5 minutes at maximum speed and the supernatant removed. The tube was washed with 500 μl of cold 80% EtOH and centrifuge at RT for 5 minutes at maximum speed The supernatant was removed and the pellet air dried for approx. 15 minutes The pellet was resuspended in 1.8 μl of DEPC H₂O per μg mRNA and used for in vitro transcription as described below.

The optimized Example 2A improved (increased) the ratio of longer second strands/shorter second strands when compared to the un-optimized Example 2.

### EXAMPLE 3

### cDNA Synthesis from Total RNA Using Random Nonamer Primers

RNA Isolation

RNA was isolated using the RNeasy kit (Qiagen) using the manufacturer's recommended conditions as described in Example 1.

First Strand cDNA Synthesis

This procedure was carried out as described in Example 1.

Second Strand Synthesis Procedure (Example 3)

The sample was placed on ice, and 2.5 μg of random nonamers per μg of initial total RNA was added. The sample was mixed, spun, and incubated at 95° C. for 1 minute, followed by rapid cooling to 4° C. After chilling for one minute, the sample was spun at 4° C. to collect condensation. A master mix was prepared containing, the following:

30 μl 5× Second strand Buffer

3 μl [10 mM] dNTP Mix

1 μl [10U/μl] E.coli DNA Ligase (optional)

4 μl [10U/μl] E.coli DNA Polymerase I

DEPC H20 to a volume of 130 μl

This master mix (130 μl) was added to the first strand synthesis reaction, and the sample incubated at 16° C. for 2 hours. Then 2 μl [10U] of T4 DNA polymerase was added, and the sample cooled for 5 minutes at 16° C., followed by addition of 10 μl 0.5M EDTA. This sample was purified using PLG tubes as described in Examples 1 and 2.

An optimized Example 3A was performed in the same manner as the previous optimized Examples 1A and 2A, and the optimized Example 3A improved (increased) the ratio of longer second strands/shorter second strands when compared to the un-optimized Example 3.

### EXAMPLE 4

### in vitro Transcription and Labeling from cDNA Using RNA Polymerase

The procedure described below uses the following reagents (suppliers shown in parentheses): T7 Megascript Kit (Ambion); RNeasy Mini Kit (Qiagen); Bio-11-CTP (Enzo Biochem); Bio-16-UTP (Enzo); β-mercaptoethanol (Sigma); Ethanol (200 proof) (Warner-Graham): DEPC H₂O (Quality Biological).

An NTP Labeling Master Mix was prepared, containing enough reagent for 4 reactions:

8 μl T7 10× ATP [75 mM]

8 μl T7 10× GTP [75 mM]

6 μl T7 10× CTP [75 mM]

6 μl T7 10× UTP [75 mM]

15 μl Bio-11-CTP [10 mM]

15 μI Bio-16-UTP [10 mM]

In vitro transcription (IVT) was carried out using the T7 Megascript System (Ambion). For each reaction a master mix of the following reagents was combined at room temperature:

1× Reaction

14.5 μl NTP labeling mix

2.0 μl 10× T7 Transcription Buffer

2.0 μl double-stranded cDNA (approx. 1 μg to 1.9 μg)

2.0 μl 10× T7 enzyme mix

The mixture was incubated at 37° C. for 6 hours and then purified using RNeasy columns (Qiagen) as follows: B-ME (10 μB-ME per 1 ml) was added to Buffer RLT before use, and 4 vols. of 100% EtOH then were added to Buffer RPE before initial use. The sample volume was adjusted to 100 μl with RNase-free H₂O. Buffer RLT (350 μl) was added to the sample and the solution mixed well. To this solution was added 250 μl of 100% EtOH to the sample followed by mixing by pipetting. The sample (approx. 700 μl) was added to the RNeasy column which was spun at RT for 15 seconds at 10,000 rpm. The eluate was collected and run over the column once more as described above. The sample was transferred to a new collection tube and 500 μl of buffer RPE was added. The column was spun at RT for 15 seconds at 10,000 rpm to wash. An additional 500 μl of buffer RPE was loaded onto the column and spun for 10 minutes at maximum speed to dry the membrane within the column. The column was transferred to a new 1.5 ml collection tube and 50 μl of DEPC H₂O was added directly onto the membrane. The column was incubated for 1 minute and spun for 5 minutes at 10,000 rpm to elute. An additional 50 μl of DEPC H₂O was added and the column incubated and spun again. The final volume collected was 100 μl. The O.D. of the solution was recorded and 280/260 ratio used to calculate the concentration of aRNA present. The sample also was checked on an agarose gel.

The RNA then was fragmented in preparation for analysis on a DNA microarray (DNA chip). The minimum concentration for aRNA for this step must be 0.6 μg/μl. Fragmentation buffer (5× solution: 200 mM Tris-acetate pH 8.1, 500 mM KOAc, 150 mM MgOAc, in RNase Free water) was added (¼ volume of 5× fragmentation buffer to the total volume of unfragmented aRNA). The reaction mixture was incubated at 94° C. for 35 minutes, and then cooled on ice. The resulting sample was used for analysis on the DNA micro array.

### EXAMPLE 5

### Double in vitro Transcription with Nonamer in Both Rounds

RNA Isolation

Total RNA can be prepared as follows:

1,000 or 10,000 cells are aliquotted into low-adhesion 1.5 ml Eppendorf tubes with 0.25 ml complete medium. 3 μl glycogen (20 μg/μl) is then added to each tube Next, 0.75 ml of TRIzon LS reagent is added to each tube and pipetted 5 times to mix.

0.2 ml chloroform is then added to each tube and mixed by tapping the tube. The tubes are then spun 10 min at approximately 10,000-13,000×g. The sample is then precipitated with alcohol. The sample is air-dried and resuspend in 10 μl RNAsecure (Ambion). RNA integrity is then checked by electrophoresis (FMC).

### First Round Amplification

cDNA Synthesis

The RNA above is then subjected to a first round of amplification as follows:

cDNA is synthesized by mixing cellular RNA obtained as above with 1 μl of 100 pM/μl T7-oligodT primer. Example 1 above is followed for first strand cDNA synthesis. Example 3 above is followed for second strand cDNA synthesis except that 2.5 μg 9-mer is added per μg total RNA (e.g., 25 μg for 10 μg RNA, 5 μg for 1 μg or 100 ng RNA). The mixture is heated at 95° C. for 1 min and then rapidly cooled. 2 μl (10U) of T4 DNA polymerase (Life Technologies, Gaithersburg, Md.) is added and the tube is incubated for 10 min at 16° C. The sample is then subjected to a phenol/chloroform extraction using PLG Tubes (1.5 ml) and precipitate with 2.5 vol ethanol. The pellet is washed with 0.5 ml 80% ethanol and resuspended in 8 μl DEPC-water.

In Vitro Transcription Reaction

The cDNA above is then in vitro transcribed using the MEGAscript™ kit (Ambion, Austin, Tex.) by adding the following in order: 2 μl 10× ATP, 2 μl 10× CTP, 2 μl 10× UTP, 2 μl 10× GTP, 2 μl 10× T7 enzyme buffer, 8 μl amplified cDNA template, and 2 μl 10× T7 enzyme mix for a total volume 20 μl. The sample is mixed well and incubated, for 6 hours at 37° C. followed by a continuous 4° C. incubation in a thermocycler (from MJ).

Transcribed RNA is cleaned-up with an RNeasy kit (Qiagen, Valenci, Calif.) or alternatively cleaned-up with a Zymo Kit (Zymo Research, Orange, Calif.), omitting the EtOH ppt. step. RNA is eluted with 2× RNase-free water and precipitated with 2.5 vol ethanol. The pellet is washed with 0.5 ml 80% ethanol, air-dried, and resuspended in 6 μl DEPC-water.

### Second Round Amplification

Second cDNA Synthesis

The transcribed RNA above is then subjected to a second round of amplification as follows: Mix 6 μl aRNA with 5 μl 5 μg/μl random 9-mer. Incubate at 70° C. for 10 min. Chill on ice/water slurry for at least 1 min. Mix with 4 μl 5× first-strand buffer, 2 μl 0.1M DTT and 1 μl 10 mM dNTP, and incubate at 42° C. for 2 min. Add 1 μl Superscript II (Life Technologies, Gaithersburg, Md.), 1 μl of E. coli DNA Polymerase (Life Technologies, Gaithersburg, Md.) and incubate at 37° C. for 1.5 hr. Add 1 μl (2U) RNaseH (Life Technologies, Gaithersburg, Md.) and incubate at 37° C. for 20 min. Heat at 95° C. for 1 min, then chill on water/ice slurry for 5 min.

Add 1 μl 100 pM/μl T7-promoter region containing primer, SEQ. ID No. 1, (Operon Technologies, Alameda, Calif.) and incubate at 70° C. for 5 min and then 42° C. for 10 min. Add 30 μl 5× second-strand buffer [Life Technologies, Gaithersburg, Md.], 3 μl 10 mM dNTP, 4 μl E. Coli DNA polymerase I, 1 μl RNaseH, and 90 μl RNAse-free water. Incubate at 16° C. for 2 hrs. Add 2 μl T4 DNA polymerase, incubate at 16° C. for 10 min. The sample is then phenol/chloroform extracted in a PLG tube, and precipitated with ethanol. The pellet is washed with 80% ethanol and resuspended in 8 μl RNase-free water.

Second In Vitro Transcription Reaction

A second in vitro transcription reaction is performed using the above cDNA and the MEGAscript™ kit from Ambion, adding in order to make the following reaction: 2 μl 10× ATP, 2 μl 10× CTP, 2 μl 10× UTP, 2 μl 10× GTP, 3.75 μl 10 mM Bio-11-CTP(Enzo), 3.75 μl 10 mM Bio-16-UTP(Enzo), 3 μl 10× T7 enzyme buffer, 8 μl amplified cDNA template, 2 μl 10× T7 enzyme mix for a total volume 28.5 μl. The sample is mixed and incubated, for 6 hours at 37° C. followed by continuous 4° C. incubation in a thermocycler (MJ).

Synthesis is checked by running 2.5-5 μl of the reaction on a 1× MOPS gel. The rest of the RNA is cleaned-up with an RNeasy kit (Qiagen). The final volume should be 100 μl. An O.D. reading is then taken to determine the concentration of RNA.

The invention has been disclosed broadly and illustrated in reference to representative embodiments described above. Those skilled in the art will recognize that various modifications can be made to the present invention without departing from the spirit and scope thereof.

What is claimed is:

1. A method for amplifying a population of RNA molecules comprising:
(a) preparing double-stranded cDNA by:
(i) hybridizing at least one primer comprising an RNA polymerase promoter to said population of RNA molecules and extending said primer by reverse transcription to generate single-stranded cDNA, and
(ii) synthesizing double-stranded cDNA from said single-stranded cDNA by priming with an oligonucleotide mixture having a random sequence selected from the group consisting of a tetramer oligonucleotide mixture, a pentamer oligonucleotide mixture, a hexamer oligonucleotide mixture, a heptamer oligonucleotide mixture, an octamer oligonucleotide mixture, a nonamer oligonucleotide mixture, a decamer oligonucleotide mixture and mixtures thereof; and
(b) transcribing amplified copies of anti-sense RNA from said double-stranded cDNA.

2. The method of claim 1, wherein said RNA polymerase promoter is a bacteriophage T7 RNA polymerase promoter, a bacteriophage T3 RNA polymerase promoter, or a bacteriophage SP6 RNA polymerase promoter.

3. The method of claim 1, further comprising fragmenting the amplified anti-sense RNA.

4. The method of claim 3, wherein said fragmentation comprises heating the amplified anti-sense RNA at 95° C.

5. The method of claim 1, wherein said population of RNA molecules comprises poly(A)+RNA.

6. The method of claim 1, wherein said population of RNA molecules comprises total RNA.

7. The method of claim 1, wherein said at least one primer comprises the nucleotide sequence: 5′-ggc cag tga att gta ata cga ctc act ata ggg agg egg ttt ttt ttt ttt ttt ttt ttt ttt-3′ (SEQ ID NO: 1).

8. The method of claim 1, wherein said amplified RNA is labeled with a radioisotope, a chromophore, a fluorophore, an enzyme, or a reactive group.

9. The method of claim 8, wherein said amplified anti-sense RNA is labeled with a biotin moiety.

10. The method of claim 1, wherein said oligonucleotides are phosphorylated at the 5′end.

11. The method of claim 1, wherein step (ii) comprises incubating said single-stranded cDNA with a DNA ligase and a DNA Polymerase.

12. A method for amplifying a population of RNA molecules comprising:
(a) preparing a first double-stranded cDNA by:
(i) hybridizing a first primer comprising an RNA polymerase promoter to said population of RNA molecules and extending said primer by reverse transcription to generate single-stranded cDNA, and
(ii) synthesizing a first double-stranded cDNA from said single-stranded cDNA by priming with an oligonueleotide mixture having random sequence selected from the group consisting of a tetramer oligonucleotide mixture, a pentamer oligonucleotide mixture, a hexamer oligonucleotide mixture, a heptamer oligonucleotide mixture, an octamer oligonucleotide mixture, a nonamer oligonucleotide mixture, a decamer oligonucleotide mixture and mixtures thereof; and
(b) transcribing copies of antisense RNA from said first double-stranded cDNA;
(c) preparing a second double-stranded cDNA by:
(i) hybridizing a second oligonueleotide mixture having random sequence and extending said oligonueleotide mixture by reverse transcription to generate single-stranded cDNA, and
(ii) synthesizing double-stranded cDNA from said single-stranded cDNA by priming with a second primer comprising an RNA polymerase promoter; and
(d) transcribing copies of amplified RNA from said second double-stranded cDNA.

13. The method of claim 12, further comprising adding DNA polymerase in step (c)(i).

14. The method of claim 12, wherein said RNA polymerase promoter is a bacteriophage T7 RNA polymerase promoter, a bacteriophage T3 RNA polymerase promoter, or a bacteriophage SP6 RNA polymerase promoter.

15. The method of claim 12, further comprising the step of fragmenting the amplified RNA.

16. The method of claim 15, wherein said fragmentation comprises heating the amplified anti-sense RNA at 95° C.

17. The method of claim 12, wherein said population of RNA molecules is RNA from 1-10, 10-100, 100-1000, 1000-10,000, or 10,000-100,000 cells.

18. The method of claim 12, wherein said population of RNA molecules is RNA selected from biopsies, micro-dissected tissues, tissue cultures, cell cultures, flow cytometry sorted cell preparations and histological sections.

19. The method of claim 1 wherein said oligonucleotide mixture is a nonamer oligonucleotide mixture.

20. The method of claim 12 wherein said oligonucleotide mixture is a nonamer oligonucleotide mixture.


SEQUENCE LISTING (1 sequence)

SEQ ID NO: 1 - DNA, 63 bp, Artificial sequence
  T7 promoter-containing primer
ggccagtgaattgtaatacgactcactatagggaggcggtttttttttttttttttttttttt